(19) United States
(12) Patent Application Publication
(10) Pub. No.: US 2024/0253929 A1
(43) Pub. Date: Aug. 1, 2024

(54) SHEET CONVEYANCE GUIDE

(71) Applicants: KOHA Co., Ltd., Tokyo (JP); GLORY LTD., Himeji-shi (JP)

(72) Inventors: Kosuke NAGASHITA, Tokyo (JP); Toshio SUZUKI, Tokyo (JP); Yasuo SEKINE, Tokyo (JP); Hiroki ISHINO, Himeji-shi (JP); Eita TAKASHIMA, Himeji-shi (JP)

(21) Appl. No.: 18/422,918

(22) Filed: Jan. 25, 2024

(30) Foreign Application Priority Data
Jan. 27, 2023 (JP) 2023-011064

(51) Int. Cl. B65H 27/00

(52) U.S. Cl. CPC B65H 27/00; B65H 2402/44

(57) ABSTRACT

A sheet conveyance guide installed in a conveying surface of a conveying path that is configured to convey a sheet. The sheet conveyance guide includes a transparent member having a light transparency and including a surface formed to be flush with the conveying surface, and a housing member including a housing portion to house the transparent member and a surface around the housing portion formed to be flush with the conveying surface. The transparent member includes an adhesion region to adhere the transparent member to an inner wall of the housing portion of the housing member, and a stress release region provided outside of the adhesion region in a width direction of the conveying path to release a stress generated in the transparent member.

### CROSS-REFERENCE TO RELATED APPLICATIONS

The present patent application claims the priority of Japanese patent application No. 2023/011064 filed on Jan. 27, 2023, and the entire contents of Japanese patent application No. 2023/011064 are hereby incorporated by reference.

### BACKGROUND ART

A conventional contact image sensor has been proposed which prevents deformation of a conveying surface on which an irradiated object such as a scanned original copy is conveyed.

The conventional contact image sensor is comprised of a transparent member formed of a flat glass plate and having a rectangular shape in a plan view with its longitudinal direction along a direction orthogonal to the conveyance direction of the irradiated object, and a resin molded cover holding the transparent member and being provided to be flush with the transparent member as well as the conveying surface of the irradiated object, and is configured such that the transparent member is fixed to the cover by bringing a peripheral surface of the transparent member into contact with plural protrusions provided in a spatial region of the cover so as to protrude from four sides and then filling a gap around the transparent member with an adhesive.

### SUMMARY

The technique disclosed herein relates to a sheet conveyance guide configured to be installed in a conveying surface of a conveying path that conveys a sheet, the sheet conveyance guide comprising:
a transparent member comprising a light transparency and a surface formed to be flush with the conveying surface; and
a housing member comprising a housing portion to house the transparent member and a surface around the housing portion formed to be flush with the conveying surface,
wherein the transparent member comprises an adhesion region to adhere the transparent member to an inner wall of the housing portion of the housing member, and a stress release region provided outside of the adhesion region in a width direction of the conveying path to release a stress generated in the transparent member.

### BRIEF DESCRIPTION OF DRAWINGS

FIG. 1 is a perspective view showing an example of an appearance of a light irradiation device in an embodiment of the present embodiment.

FIG. 2A is a cross-sectional view taken along line A-A in FIGS. 1 and 3.

FIG. 2B is an enlarged view of a portion B of FIG. 2A.

FIG. 3 is a plan view showing a sheet conveyance guide shown in FIG. 1.

FIG. 4 is a perspective view showing essential part of the sheet conveyance guide shown in FIG. 1.

FIG. 5 is a cross-sectional view taken along line C-C in FIG. 3.

FIG. 6 is a perspective view showing a guide member.

FIGS. 7A-7C show explanatory cross-sectional views showing a process of adhering a transparent member to the guide member as viewed in an X direction.

FIG. 8 is a perspective view showing the guide member in Modification 1.

### DESCRIPTION OF EMBODIMENTS

In the conventional contact image sensor, the conveying surface is flattened by filling the gap between the transparent member and the cover so as to fill up the gap portion. However, if the transparent member is deformed by changes in ambient temperature, the deformation of the conveying surface or cracks in the transparent member may be caused due to a difference in linear expansion coefficient between the transparent member and the cover.

It is an object of the embodiment to provide a sheet conveyance guide that can suppress deformation of a sheet conveying surface or cracking of a transparent member even under changes in ambient temperature while ensuring flatness of the sheet conveying surface.

Embodiments will be described below in reference to the drawings. Constituent elements having substantially the same functions are denoted by the same reference numerals across the drawings and overlapping explanation will be omitted. In addition, although an example of applying the sheet conveyance guide to the light irradiation device will be described in the present embodiment, the application is not limited thereto.

### Embodiment

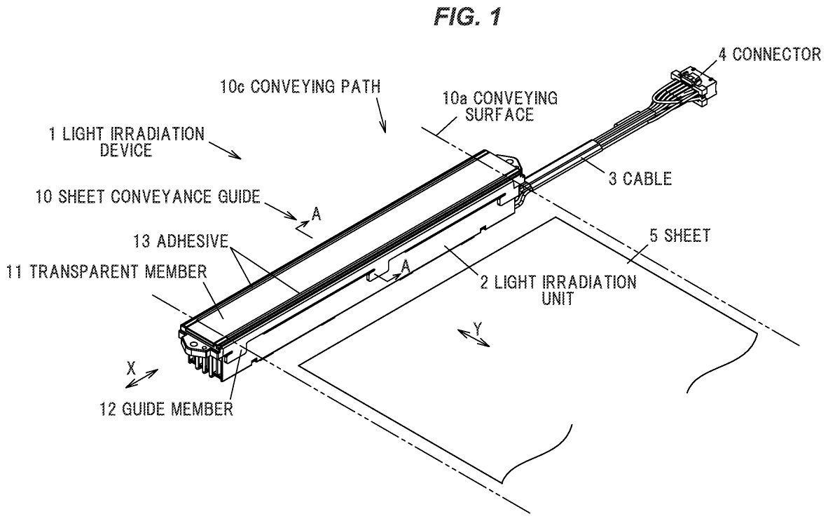

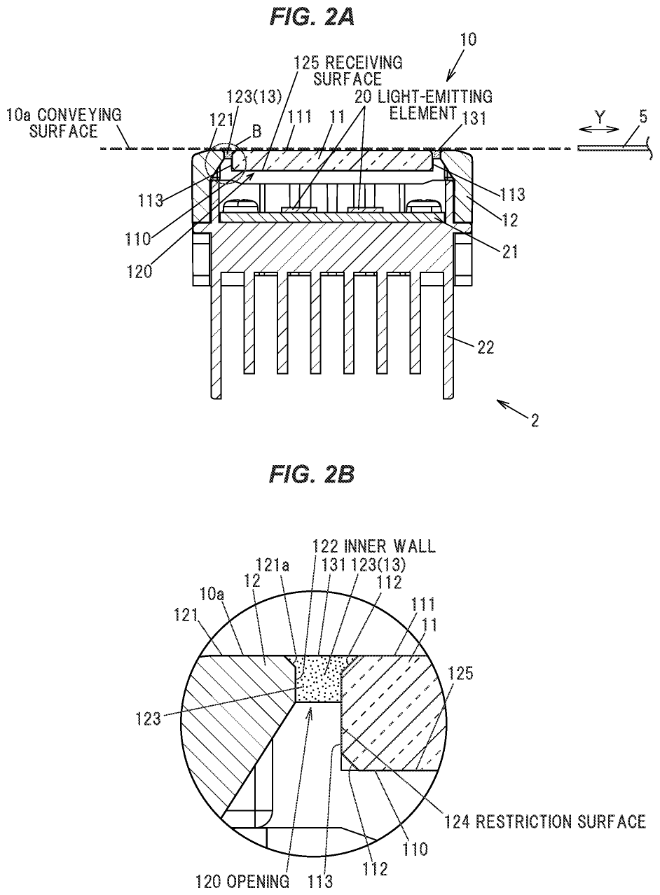

FIG. 1 is a perspective view showing an example of an appearance of a light irradiation device in the embodiment. A light irradiation device 1 is comprised of a light irradiation unit 2 to irradiate a sheet 5, which is an object to be conveyed, with light emitted from plural light-emitting elements 20 (see FIG. 2), and a sheet conveyance guide 10 that is combined with the light irradiation unit 2. A cable 3 to supply power to the light-emitting elements 20 extends out of the light irradiation unit 2, and a connector 4 is connected to an end of the cable 3. In the present specification and drawings, Y represents a conveyance direction of the sheet 5 (the lateral direction of the transparent member, also referred to as the Y direction), and X represents a direction parallel to a conveying surface 10a and orthogonal to the conveyance direction Y (the longitudinal direction of the transparent member, also referred to as the X direction). The light-emitting element 20 is an example of a photonic device.

The sheet 5 is, e.g., paper (bills, manuscripts, recording paper, paper sheets, photographs, etc.), or plastic film, etc., but is not limited thereto. The conveying surface 10a is formed along the Y direction, and the sheet 5 is conveyed along the conveying surface 10a of a conveying path 10c. The sheet 5 may be conveyed unidirectionally or bidirectionally in the Y direction.

The sheet conveyance guide 10 includes a plate-shaped transparent member 11 having light transparency, and a guide member 12 that holds the transparent member 11 while allowing the transparent member 11 to be exposed and guides the sheet 5 in the conveyance direction Y, and the sheet conveyance guide 10 has a structure in which the transparent member 11 is fixed to the guide member 12 with an adhesive 13. The guide member 12 is an example of a housing member.

FIG. 2A is a cross-sectional view taken along line A-A in FIGS. 1 and 3, and FIG. 2B is an enlarged view of a portion B of FIG. 2A. The light irradiation unit 2 includes a substrate 21 on which the plural light-emitting elements 20 are mounted along the X direction, and a heat sink 22 that is arranged on a back side of the substrate 21 and releases heat generated by the light-emitting elements 20 to the outside.

The light-emitting element 20 maybe a light-emitting diode (LED) or a laser diode (LD). When the sheet conveyance guide 10 is applied to an image reading device, the light-emitting elements 20 and light-sensitive elements may be used as the photonic device. Moreover, as the photonic device, light-sensitive elements may be used instead of the light-emitting elements 20.

The transparent member 11 is formed of a material having transparency to light emitted from the light-emitting elements 20, e.g., glass, or a resin or plastic such as acrylic or polycarbonate. When, e.g., ultraviolet light-emitting diodes (LED) are used as the light-emitting elements 20 to sterilize the sheet 5, a material having excellent ultraviolet transparency, e.g., quartz glass or ultraviolet ray transmitting glass, etc. is preferably used as the material of the transparent member 11.

The guide member 12 is formed of, e.g., a resin such as polycarbonate. The guide member 12 may be formed of a material having a linear expansion coefficient close to or the same as that of the transparent member 11. This further suppresses deformation of the sheet conveying surface 10a or cracking of the transparent member 11 under changes in ambient temperature.

The adhesive 13 is applied to fill adhesive filling portions 123 (described later) to adhere the transparent member 11 to the guide member 12. A thickness of the adhesive filling portion 123 in a direction orthogonal to the X direction and the Y direction does not need to be a similar thickness to the transparent member 11 and may be not more than ½, or not more than ⅓, of the thickness of the transparent member 11. An ultraviolet-curable adhesive, a thermosetting adhesive, or a cold-setting adhesive, etc. can be used as the adhesive 13. When an ultraviolet-curable adhesive is used as the adhesive 13, the adhesive 13 is cured by exposure to ultraviolet rays. When a thermosetting adhesive is used as the adhesive 13, a heating furnace is used for curing.

### Description of the Conveying Surface

Next, the conveying surface 10a will be described in reference to FIGS. 1. and 2. The conveying surface 10a is a region on the conveying path 10c where the sheet 5 is conveyed (a region defined by dashed-double dotted lines in FIG. 1). The sheet 5 is conveyed within the region defined by the dashed-double dotted lines in FIG. 1 in a normal conveyance state, but the sheet 5 maybe conveyed in a state of being partially located out of the region defined by the dashed-double dotted lines in FIG. 1. The transparent member 11 has a surface 110 (hereinafter, referred to as the “back surface”) on the inner side and a surface 111 (hereinafter, referred to as the “front surface”) on the outer side, and is formed such that the front surface 111 is flush with the conveying surface 10a conveying the sheet 5. To enhance adhesion with the guide member 12, the transparent member 11 also has a chamfered portion 112 at which a corner is chamfered.

The guide member 12 has an opening 120 to house the transparent member 11, and receiving surfaces 125, and is formed such that a front surface 121 around the opening 120 is flush with the conveying surface 10a. To enhance adhesion with the transparent member 11 and to smoothly convey the sheet 5, the guide member 12 also has a chamfered portion 121a at which a corner is chamfered, in a similar manner to the transparent member 11.

The sheet 5 maybe conveyed while being tilted relative to the conveyance direction Y or being shifted in the X direction, hence, a width of the conveying surface 10a in the X direction is set larger than a width of the sheet 5 in the X direction.

After the transparent member 11 is adhered to the guide member 12, it is preferable that the front surface 111 of the transparent member 11, the front surface 121 of the guide member 12, and a front surface 131 of the adhesive 13 be flat on the inner side of the conveying surface 10a such that the sheet 5 is not caught during being conveyed. That is, it is preferable that raised portions protruding from the conveying surface 10a, recessed portions recessed from the conveying surface 10a, or steps be not present.

### Configuration of Each Part

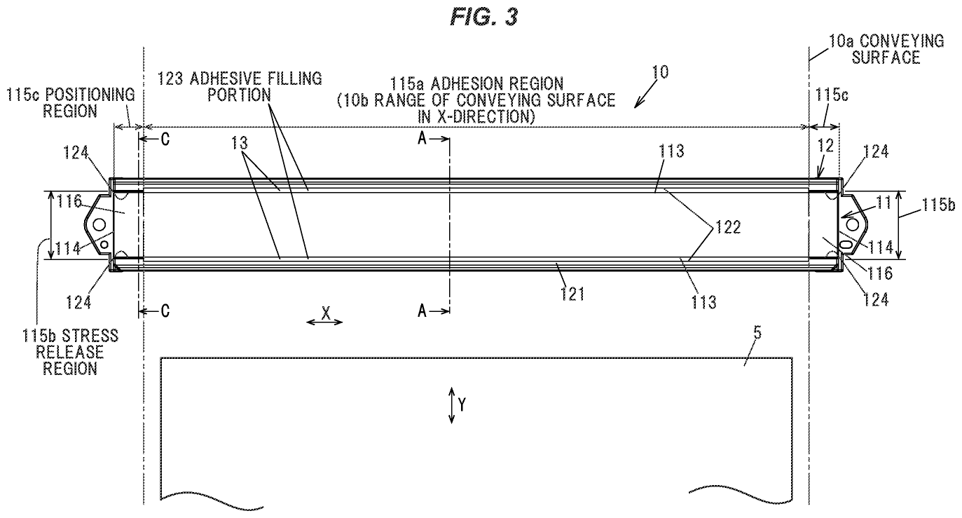

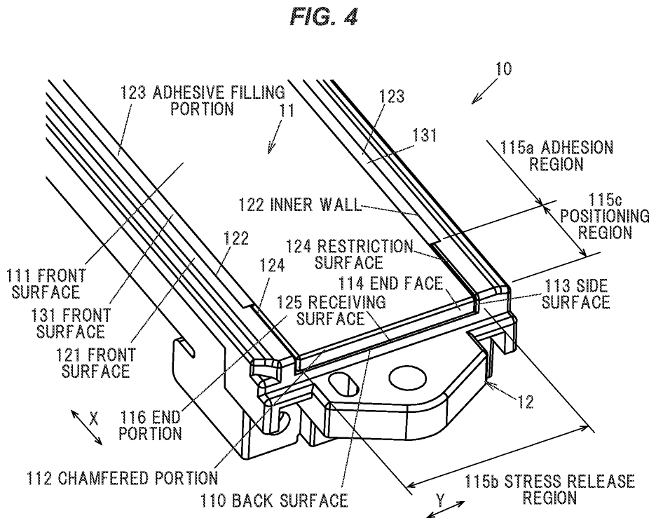

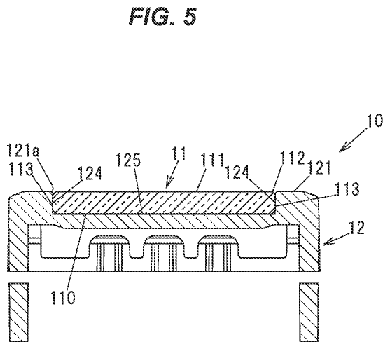

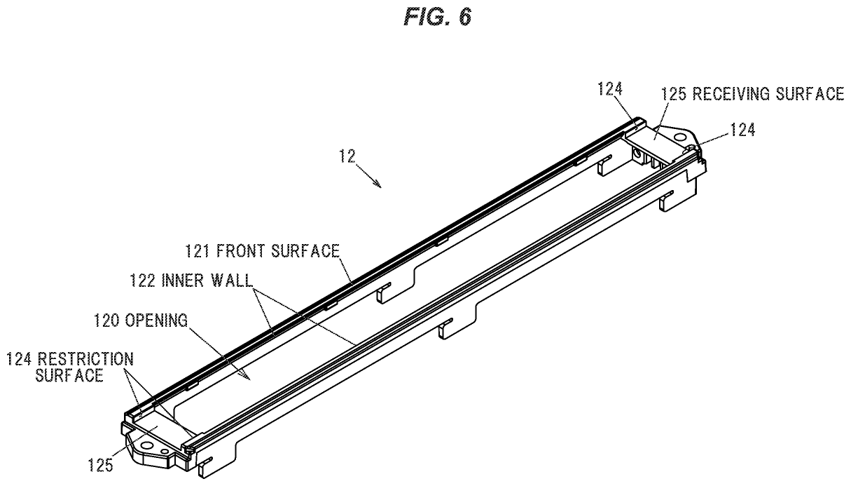

Next, the configuration of each part will be further described in reference to FIGS. 3 to 6. FIG. 3 is a plan view showing the sheet conveyance guide 10 shown in FIG. 1. FIG. 4 is a perspective view showing essential part of the sheet conveyance guide 10 shown in FIG. 1. FIG. 5 is a cross-sectional view taken along line C-C in FIG. 3. FIG. 6 is a perspective view showing the guide member 12.

The transparent member 11 has a rectangular shape in plan view that has a longitudinal direction along a direction orthogonal to the conveyance direction Y of the sheet 5. The direction orthogonal to the conveyance direction Y of the sheet 5 is a width direction of the conveying path 10c. The transparent member 11 has an adhesion region 115a, and stress release regions 115b provided outside of the adhesion region 115a in the width direction of the conveying path 10c. The transparent member 11 may further have positioning regions 115c provided outside of the adhesion region 115a in the width direction of the conveying path 10c. The adhesion region 115a may be provided inside the conveying surface 10a, with the stress release regions 115b and the positioning region 115c outside the conveying surface 10a.

The adhesion region 115a is a region where the adhesive filling portions 123 in gaps between both lateral side faces 113 of the transparent member 11 and an inner wall 122 of the opening 120 of the guide member 12 is filled with the adhesive 13 and the transparent member 11 is thereby adhered (joined) to the guide member 12. The adhesion region 115a may be provided in the same range as a range (conveyance range) 10b of the conveying surface 10a in the X-direction, or may be provided in a range slightly wider than the range (conveyance range) 10b. Since the cross-sectional shape of the adhesive filling portion 123 is the same throughout the longitudinal direction, the adhesive 13 easily flows to an edge portion (the chamfered portion 112) of the transparent member 11 on the front surface 111 side and to an edge portion (the chamfered portion 121a) of the guide member 12 on the front surface 121 side.

The stress release region 115b is provided in at least one of two longitudinal end faces 114 of the transparent member 11 such that it is not adhered to the guide member 12 and is not contacted with the guide member 12. The stress release regions 115b may be provided on both the longitudinal end faces 114 of the transparent member 11. It is thereby possible to release the stress generated in the transparent member 11 when the ambient temperature changes and the transparent member 11 is deformed in the longitudinal direction.

The positioning region 115c is a region where the lateral side faces 113 of the transparent member 11 at both longitudinal end portions 116 are in contact with restriction surfaces 124 (described later) of the guide member 12 and the position of the transparent member 11 in the lateral direction is thereby restricted. This allows positioning of the transparent member 11 in the lateral direction.

It is possible to ensure flatness of the conveying surface 10a by providing the stress release regions 115b and the positioning regions 115c outside the conveying surface 10a. That is, it is possible to ensure that the front surface 111 of the transparent member 11, the front surface 121 of the guide member 12 and the front surface 131 of the adhesive 13 filling the adhesive filling portions 123 are flat in the range 10b of the conveying surface 10a in the X-direction.

The guide member 12 includes the adhesive filling portions 123 corresponding to the conveying surface 10a, the restriction surfaces 124 restricting the position of the transparent member 11 in the Y direction, and a pair of receiving surfaces 125 in contact with the back surface 110 of the transparent member 11 at the both end portions 116. A length in the X direction over which the back surface 110 of the transparent member 11 and the receiving surface 125 of the guide member 12 are in contact with each other is set to a predetermined length (e.g., not less than 5 mm or 10 mm), thereby allowing desired dust-tight performance to be achieved. The opening 120 and the receiving surface 125 are examples of the housing portion to house the transparent member 11. The restriction surface 124 is an example of the restricting portion.

### Adhering Process

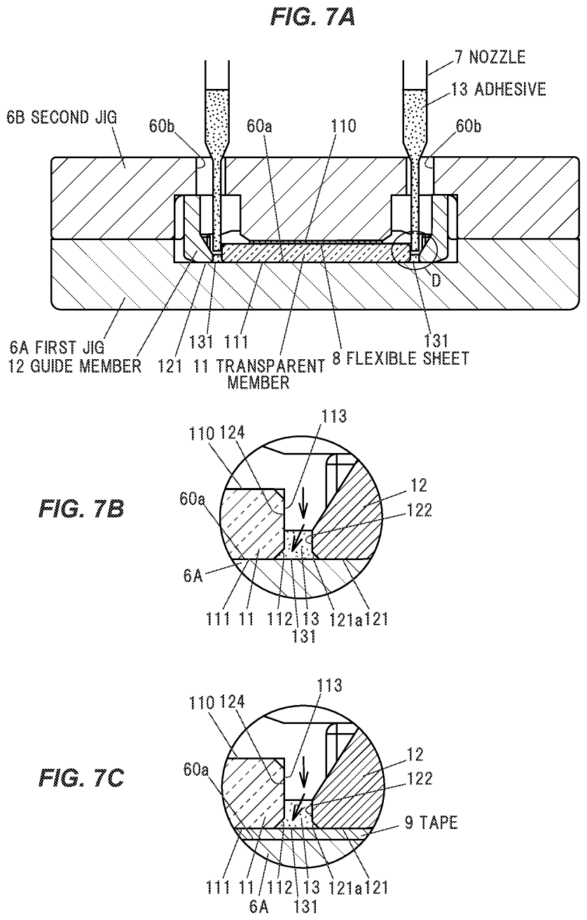

Next, an adhering process of adhering the transparent member 11 to the guide member 12 will be described in reference to FIGS. 7A-7C. FIGS. 7A-7C show explanatory cross-sectional views showing a process of adhering the transparent member 11 to the guide member 12 as viewed in the X direction, wherein FIG. 7A is a cross-sectional view showing a state in which the transparent member 11 and the guide member 12 are sandwiched between a first jig 6A and a second jig 6B, FIG. 7B is an enlarged view of a portion D in FIG. 7A, and FIG. 7C is an enlarged view of the portion D in a modification of FIG. 7B.

Before the adhering process, the transparent member 11 and the guide member 12 are placed on the first jig 6A such that the front surface 111 of the transparent member 11 and the front surface 121 of the guide member 12 are in contact with an upper surface 60a of the first jig 6A. In this regard, a PTFE (fluoroplastic) tape 9 maybe applied to the surfaces to be flush with the conveying surface 10a as shown in FIG. 7C such that the adhesive 13 does not stick to the upper surface 60a of the first jig 6A. When an ultraviolet-curable adhesive is used as the adhesive 13, a cellophane tape or a PP tape formed of polypropylene (PP), etc., which has transparency to ultraviolet rays, may be used as the tape 9. Since the front surface 111 of the transparent member 11, the front surface 121 of the guide member 12 and the front surface 131 of the adhesive 13 are formed in the same plane, it is possible to ensure the flatness of the conveying surface 10a.

Next, the transparent member 11 and the guide member 12 are pressed toward the first jig 6A by the second jig 6B. At this time, to suppress damage such as scratches or cracks on the transparent member 11, it is preferable to place a flexible sheet 8 so as to interpose between the transparent member 11 and the second jig 6B. By attaching the flexible sheet 8 to the second jig 6B, it is possible to eliminate work of attaching the flexible sheet 8 to the transparent member 11.

In this regard, the following methods are also available: a method in which the transparent member 11 is brought into contact with the upper surface 60a of the first jig 6A by the weight of the transparent member 11; and a method in which a hole for vacuum suction is provided on the first jig 6A and the transparent member 11 is brought into contact with the upper surface 60a of the first jig 6A by suction through this hole. In this case, the pressing of the transparent member 11 by the second jig 6B can be eliminated.

The second jig 6B has openings 60b formed along the X direction at positions corresponding to the adhesive filling portions 123 such that the nozzles 7 of a liquid dispensing device (dispenser) to drip the adhesive 13 can be inserted through the openings 60b.

Next, a predetermined amount of the adhesive 13 is poured into the adhesive filling portions 123 from the nozzles 7 while moving the nozzles 7 in the X direction. After that, the adhesive 13 is cured. The transparent member 11 is thereby adhered to the guide member 12.

### Functions and Effects of the Present Embodiment

The following functions and effects are exerted in the present embodiment.
(a) The stress release regions 115b of the transparent member 11 are provided outside of the conveying surface 10a. Therefore, even if the transparent member 11 and the guide member 12 are formed of different materials, it is possible to release the stress generated in the transparent member 11 due to the difference in linear expansion coefficient when the transparent member 11 is deformed in the longitudinal direction due to changes in the ambient temperature. Deformation of the sheet conveying surface 10a or cracking of the transparent member 11 can thereby be suppressed.
(b) Since the positioning regions 115c of the transparent member 11 are provided outside of the conveying surface 10a, it is possible to position the transparent member 11 in the lateral direction in the adhering process, thereby suppressing looseness. It is possible to fill the adhesive filling portions 123 in the positioning regions 115c of the transparent member 11 with the adhesive 13 in this state and thereby fix the transparent member 11 to the guide member 12. That is, it is possible to ensure the flatness of the conveying surface 10a by positioning outside the conveyance range (the conveying surface 10a). In addition, since it is not necessary to provide positioning protrusions in the adhesion range (in the adhesive filling portions 123 in the adhesion region 115a), it is possible to stably perform application work and to equalize the adhesive cross section of the adhesive filling portions 123.
(c) Since the transparent member 11 extends in the X direction to the outside of the conveying surface 10a, the adhesive 13 which needs to be controlled in terms of steps, etc. is not present at the portions of the transparent member 11 on the end portion 116 side, hence, process control can be simplified.

### Modification 1

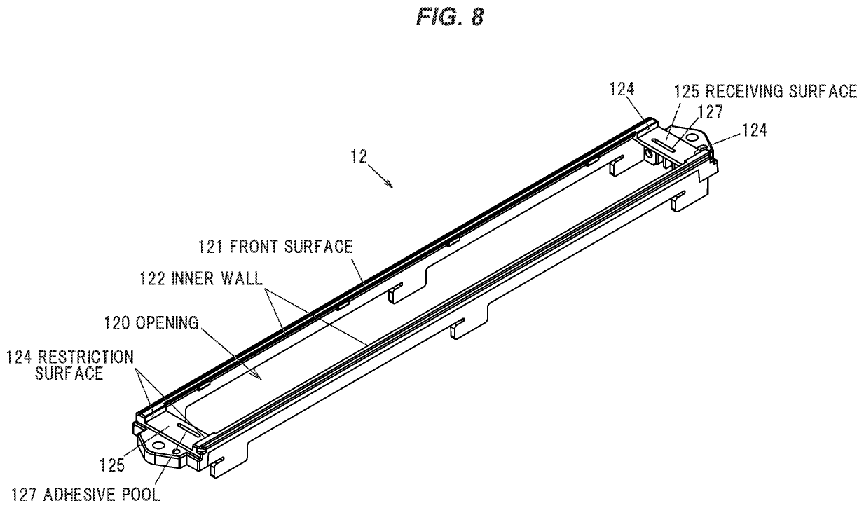

FIG. 8 is a perspective view showing the guide member 12 in Modification 1. In Modification 1, adhesive pools 127 formed by through-holes are respectively provided on the both receiving surfaces 125 of the guide member 12. By filling the adhesive filling portions 123 with the adhesive 13 and also filling the adhesive pools 127 with the adhesive 13 in the adhering process, adhesion between the back surface 110 of the transparent member 11 and the receiving surfaces 125 is improved, which further enhances dust-tight performance.

### Modification 2

Although the position of the transparent member 11 in the lateral direction is restricted by the restriction surfaces 124 of the guide member 12 in the embodiment described above, the position of the transparent member 11 in the lateral direction may be restricted by one or plural protrusions instead of using the restriction surfaces 124. In this case, frictional resistance is reduced since the contact area between the protrusion and the side face 113 in the positioning region 115c is small, hence, the transparent member 11 when expanding is easily deformed in the longitudinal direction and it is thus easier to release the stress generated in the transparent member 11.

### Example

Based on JIS D 0207 (General rules of dust test for automobile parts), a dust-tight test was conducted as follows.

### (1) Configuration in Example

The transparent member 11 with a length of 218 mm in the longitudinal direction, a width of 20 mm in the lateral direction and a thickness of 2 mm was used, the length of the adhesive filling portions 123 in the longitudinal direction was 190 mm, the guide member 12 having the adhesive pools 127 (Modification 1) was used, and the remaining configuration was the same as in the embodiment.

### (2) Test Conditions

Amount of dust: 1 kg (dust concentration: 1000 mg/m³)
Blowing time: 5 seconds
Pause time: 15 minutes
Test duration: 72 hours
Type of dust: Kanto Loam Class 8

### (3) Judgment Items and Judgment Criteria

No ingress of dust that would have a negative effect on illumination intensity.

### (4) Results of Judgment

Dust which would have a negative effect on illumination intensity was not observed visually and under microscope, and the result was pass.

### Other Embodiments

Although the embodiment has been described, the technique disclosed herein is not limited to the embodiment described above and various modifications can be implemented. For example, the case where the sheet conveyance guide 10 is applied to the light irradiation device 1 to irradiate the sheet 5 with light has been described in the embodiment, the sheet conveyance guide 10 may be applied to an image reading device that irradiates the sheet 5 with light and reads an image of the sheet 5 from the reflected light. In addition, some of the constituent elements in the embodiment described above may be omitted or changed.

### INDUSTRIAL APPLICABILITY

The technique disclosed herein is applicable to not only optical devices such as light irradiation devices, but also to various devices that convey sheets, e.g., image forming devices such as printers, copiers, facsimiles and multifunction machines, image reading devices such as document scanners, paper sheet processing devices such as currency-counting machines and automated teller machines (ATM), and sterilization devices that sterilize sheets such as bills, etc.

1. A sheet conveyance guide installed in a conveying surface of a conveying path that is configured to convey a sheet, the sheet conveyance guide comprising:
a transparent member having a light transparency and comprising a surface formed to be flush with the conveying surface; and
a housing member comprising a housing portion to house the transparent member and a surface around the housing portion formed to be flush with the conveying surface,
wherein the transparent member comprises an adhesion region to adhere the transparent member to an inner wall of the housing portion of the housing member, and a stress release region provided outside of the adhesion region in a width direction of the conveying path to release a stress generated in the transparent member.

2. The sheet conveyance guide according to claim 1, wherein the transparent member further comprises a positioning region provided outside of the adhesion region in the width direction of the conveying path to position the transparent member.

3. The sheet conveyance guide according to claim 1, wherein the transparent member is a plate which has in plan view a rectangular shape whose longitudinal direction is orthogonal to a conveyance direction of the sheet, and
wherein the stress release region is provided on at least one of two longitudinal end faces of the transparent member.

4. The sheet conveyance guide according to claim 3, wherein a positioning region to position the transparent member is provided on lateral end faces of both longitudinal end portions of the transparent member, and wherein the housing member comprises a restricting portion that is in contact with the positioning region and restricts a position of the transparent member in the lateral direction.

5. The sheet conveyance guide according to claim 4, wherein the housing portion comprises a receiving surface that is in contact with a back surface of the both longitudinal end portions of the transparent member at a position corresponding to the positioning region and has a predetermined length in the longitudinal direction of the transparent member.

6. The sheet conveyance guide according to claim 5, wherein the housing member comprises an adhesive pool on the receiving surface, and wherein the back surface of the transparent member is adhered at the both longitudinal end portions by an adhesive collected in the adhesive pool.

7. The sheet conveyance guide according to claim 1, wherein the transparent member is formed of a glass, and wherein the housing member is formed of a resin.
